(19) United States
(12) Patent Application Publication
(10) Pub. No.: US 2005/0107791 A1
(43) Pub. Date: May 19, 2005

(54) Intramedullary locked compression screw for stabiliziation and union of complex ankle and subtalar deformities

(76) Inventor: Easton L. Manderson, Ashton, MD (US)

Correspondence Address: ALFRED A. STADNICKI;ANTONELLI, TERRY, STOUT & KRAUS, LLP, SUITE 1800, 1300 NORTH SEVENTEENTH STREET, ARLINGTON, VA, 22209, US

(21) Appl. No.: 10/706,921

(22) Filed: Nov. 14, 2003

(51) Int. Cl. A61B 17/58

(52) U.S. Cl. 606/62

(57) ABSTRACT

An intramedullary lockable compression screw for stabilization and reconstruction of deformities of the ankle and subtalar joints, and which is particularly suitable for stabilization and fusion of the tibiotalar, talocalcaneal, tibiocalcaneal and/or tibiotalocalcaneal, is provided. The compression screw includes an elongated tubular member extending along a substantially straight first longitudinal axis between the leading end and the trailing end of the member. The tubular member includes a threaded leading end portion proximate the leading end, a threaded trailing end portion proximate to the trailing end, and an unthreaded shaft portion interconnecting the threaded leading end and the threaded trailing end portions. The diameter of the threaded leading end portion is smaller than the diameter of the threaded trailing end portion. A through-hole extends along a straight second longitudinal axis between a first opening, in a first area of an outer periphery of the tubular member proximate to the trailing end, and a second opening, in a second area of the outer periphery of the tubular member distal to the trailing end. The through hole is formed such that the second longitudinal axis intersects the first longitudinal axis at an angle of other than 90 degrees and so as to accommodate a locking screw.

### TECHNICAL FIELD

The present invention relates generally to the stabilization and reconstruction of deformities of body joints, and more particularly to double-threaded screws used for stabilization and reconstruction of deformities of the ankle and subtalar joints, and which are particularly suitable for stabilization and fusion of the tibiotalar, talocalcaneal, tibiocalcaneal and/or tibiotalocalcaneal joints.

### BACKGROUND OF INVENTION

Severe pain, instability, deformity and/or difficulty walking or bearing weight on a joint or joints are usually secondary to painful traumatic arthritis, neuropathic, destruction of joints, post infection arthritis, failed joint replacement, especially of the ankle and are commonly caused by garden variety primary osteoarthritis or degenerative arthritis. Such pain, instability, deformity and/or difficulty walking or bearing weight, typically indicate that fusion in the ankle or subtalar joints is required.

A wide variety of traditional techniques and devices have been used to effect fusion in the foot and ankle, such as external fixators, crossed large internal screws passed from one bone to another, metal plates that pass from one bone to the another and held in place with screws to each involved bone, or cortical bone grafts, allografts or autografts that bridge the involved bones and are placed with or without screws fixing the graft to each involved bone. Generally, to fuse the joints, e.g. the tibiotalar, talocalcaneal, tibiotalocalcaneal or tibiocalcaneus jioint, as much cartilage as possible is removed from the adjacent bones of the subject joint. These bones are then coapted or placed together and then held together tightly.

Many of the traditional techniques for fusion in the ankle and subtalar joints require that there be no weight bearing load placed on the involved joint until fusion has be achieved. This is quite difficult for many patients who have suffered trauma involving the torso, the spine and the upper extremities, since it makes it very difficult for them to balance themselves on one lower extremity using a walking aid. This is also quite difficult, or even impossible, for the frail and the elderly patients.

Furthermore, surgeons using traditional techniques for reconstruction of complex painful deformities of the ankle and/or subtalar joints, have experienced a high percentage of poor results. Often, patients have undergone multiple procedures without success leaving the soft tissue envelope compromised, and prone to infection and increased morbidity. In many of the most difficult cases, the residual pain and deformity has necessitated amputation as the final solution.

Recently, a technique using intramedullary nails has become popular. However, conventional nails require the use of elaborate jigs to precisely insert locking screws through the small holes in the nails, and thereby lock the nail in place. This makes the insertion of the locking screws very difficult for some surgeons. This is unfortunate because, although the use of intramedullary nails allows a weight bearing load to be placed on the involved joint prior to fusion being achieved, the installation procedure for is much more difficult than the procedure required by traditional techniques.

An even more recent alternative to traditional techniques for stabilization and/or amputation is coaptation across the prepared bones of the joints involved in fusion, using an intramedullary, double-threaded compression screw. This alternative is generally used in only very complex and difficult cases of arthropathy of the joints, such as those mentioned above, which must otherwise be treated by amputation, since salvage by traditional approaches are usually unsuitable or ineffective.

The procedure utilized to insert a conventional double-threaded compression screw into the intramedullary canal is quite simple. An anterior or posterior longitudinal midline incision is used to access the joint and the cartilaginous surfaces of the joint involved in fusion. Then a 1.0 inch or so transverse incision is made at the intersection of a line along the anterior border of the fibula and proceeding across the plantar surface of the foot, with a line drawn through the center of the plantar aspect of the heel. Blunt dissection is made down to the inferior surface of the calcaneus. A periosteal elevator is used to gently push aside the soft tissue from the proposed entry site, in order to insert a suitable guidewire, which will accommodate the reamer and the intramedullary double-threaded compression screw.

The guide wire is inserted from the inferior surface of the calcaneus through the calcaneus, if a talocalcaneal fusion is planned. Alternatively, the guidewire is inserted into the intramedullary area of the tibia and across the talus with the foot held in neutral position of flexion, extension, varus, valgus and rotation, if a tibial-talar fusion is planned. The intramedullary position of the guide wire is verified by intraoperative x-ray images or by fluoroscopy, thereby confirming coaptation and alignment.

The double-threaded compression screw is seated using a cannulated reamer of an appropriate size. The reamer size is dependent on the size of the extremity. The reamer is placed over the guide wire to prepare the intramedullary canals of the bones involved in the fusion. Usually, a cannulated reamer, which is slightly larger than the reamer used to prepare the tibial canal, is used to prepare the calcaneal canal entry point. After seating the double-threaded compression screw, a few threads of the trailing end of the screw will typically remain outside the calcaneus. The proximal end of the screw must engage the diaphysis of the tibia.

Rigid fixation is immediate and coaptation of the denuded surfaces is very precise. Autogenous bone grafting placed at the fusion site is generally recommended for these complex cases, although not mandatory. Allograft is also acceptable for placement at the fusion sites.

However, unlike intramedullar nails used for ankle fusion, this double-threaded compression screw does not require the use of an elaborate jig to insert one or more interlocking screws. At present, the intramedullary nails used for ankle fusion, even with interlocking screws, do not bind well in the calcaneus. Therefore, early weight bearing with these nails is not wise. Needless to say, insertion of interlocking screws, even with the elaborate jigs, is difficult for many surgeons. The use of a double threaded, intramedullary compression screw, because of its firm binding in the calcaneal cortex compared to a nail without threads, will allow early weight bearing prior to fusion being achieved. The installation procedure of the double threaded screw for ankle fusion is much easier than the procedure for conventional intramedullary nails because the interlocking screws are inserted without the use of a jig and enhance the fixation already achieved by the threads of the screw.

### OBJECTIVES

Accordingly, it is an object of the present invention to provide an improved lockable double-threaded intramedullary compression screw for stabilizing by fusion, a body joint such as the ankle and/or subtalar joint.

It is another object of the present invention to provide a lockable double-threaded intramedullary compression screw which can be locked in place by one or more locking screws without the use of an aiming jigs or other alignment device to insert a locking screw(s).

It is a further object of the present invention to provide a more easily installable implant, which can be used to salvage a lower extremity damaged ankle or subtalar joints by stabilizing the joints chosen for fusion.

It is a still further object of the present invention to provide a lockable double-threaded intramedullary compression screw which can be locked in place to achieve arthrodesis of the ankle by the insertion of one or more locking screws using palpation or direct vision.

These and other objects and advantages of the present invention will become apparent from the following description of the invention and the appended claims taken in conjunction with accompanying drawings.

### SUMMARY OF THE INVENTION

In accordance with the invention, an intramedullary lockable compression screw is provided for stabilizing a joint in a body. The compression screw has an elongated tubular member and a through-hole configured to accommodate a locking screw.

The elongated tubular member extends along a substantially straight first longitudinal axis between the leading end and the trailing end of the tubular member. The tubular member includes at least three distinct portions. A threaded leading end portion has a first diameter and is disposed proximate to the leading end. A threaded trailing end portion has a second diameter, which is larger than the first diameter, and is disposed proximate to the trailing end. An unthreaded shaft portion interconnects the threaded leading end and the threaded trailing end portions.

The through-hole is configured to accommodate a locking screw, preferably a 4 mm locking screw, and extends along a straight second longitudinal axis between first and second openings in the outer periphery of the tubular member. The second longitudinal axis intersects the first longitudinal axis at an angle of other than 90 degrees. The first opening is in a first area of the outer periphery disposed proximate to the trailing end of the tubular member. The second opening is in a second area of the outer periphery disposed distal, as compared to the first opening, to the trailing end of the tubular member.

According to an aspect of the invention, the elongated tubular member has a sleeve portion extending from the trailing end, away from the leading end. The sleeve portion is aligned with the through-hole and configured to guide the locking screw into the first opening.

Typically, the compression screw will additionally include another through-hole configured to accommodate another locking screw, also preferably of 4 mm. This other through-hole extends along a straight third longitudinal axis between third and fourth openings in the outer periphery of the tubular member. The third opening is in a third area and the fourth opening is in a fourth area of the outer periphery of the tubular member. Both the third and the fourth areas are proximate to the leading end of the tubular member. The third longitudinal axis intersects the first longitudinal axis, preferably at a 90 degree angle.

Beneficially, a cross section of this other through-hole, taken along the third longitudinal axis, has different first and second dimensions. More particularly, the first dimension, which is in a direction parallel to the first longitudinal axis and is preferably about 1 inch in length, is larger than the second dimension, which is in a direction perpendicular to the first longitudinal axis.

According to still other aspects of the invention, an aperture is provided in the trailing end of the tubular member. The aperture is configured to accommodate a tool for applying a torque to the tubular member. Preferably, the aperture includes both a non-threaded portion configured to accommodate a tool for applying a torque to the tubular member, and a threaded portion configured to engage a tool for extracting the tubular member from a body joint.

Thus, the lockable, double-threaded compression screw described herein allows early weight bearing even while healing and fusion are taking place. This reduces the nursing care required in a supervised facility or at home, since it is much easier for the patient to engage in the activities of daily living, such as going to the toilet or to a dining area, independently with or without a walking aid.

An additional benefit of the described locked double threaded compression screw is that no jigs are required to place the locking screws. This makes the operative time much shorter and the surgeon's exposure to fluoroscopic radiation during surgery much less.

Therefore, the subject double-threaded locked intramedullary compression screw for fusion is ideal for very complex cases. The procedure for placement of the locking screw(s) requires much less radiation during surgery than that required for placement of a locking screw(s) in a conventional intramedullary nail.

Furthermore, the subject intramedullary double threaded compression screw can be locked with one or more locking screws, without the jig(s) required to insert the locking screw(s) in a conventional intramedullary nail. This is because, the proximal through-hole, near the trailing end of the tubular member, is disposed and orientated so as to be visible to the surgeon's naked eye, and the distal through-hole, near the leading end of the tubular member, is in the form of an enlarged slot that is much larger than the small holes formed in conventional double-threaded compression screws. Thus, using the compression screw described herein, the locking screws can be applied proximally and/or distally using direct vision or by palpation, with or without a guidewire.

### BRIEF DESCRIPTION OF THE DRAWINGS

FIG. 1 is a longitudinal cross-sectional view of a foot and ankle with guide wire and double-threaded compression screw introduced through the calcaneus and into the tibia, in accordance with the present invention.

FIG. 2 depicts a lockable double-threaded compression screw according to the present invention.

FIG. 3 depicts a side view of the double-threaded compression screw shown in FIG. 2.

FIG. 4 depicts a cross-sectional view of the double-threaded compression screw shown in FIG. 3.

FIG. 5 depicts a view of the trailing end of the double-threaded compression screw shown in FIG. 2.

FIG. 6 depicts the trailing end of the double-threaded compression screw shown in FIG. 2, with the proximal locking screw inserted.

### PREFERRED EMBODIMENT OF THE INVENTION

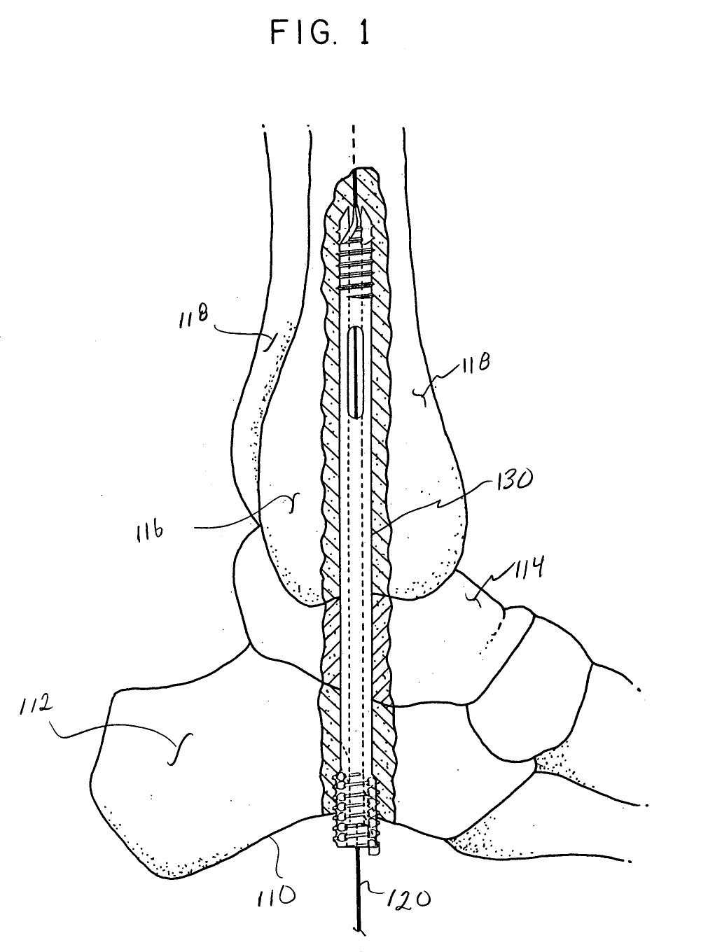

A critical step in the use of the invention is the creation of the intramedullary canal or cavity across the joints between the bones involved in the fusion. As shown in FIG. 1, the canal extends from the sole of the foot 110, through the calcaneus 112, and talus 114, and tibia 118. The canal must be reamed to a sufficient size to accept a guide wire 120 and to accommodate the lockable double-threaded intramedullary compression screw 130.

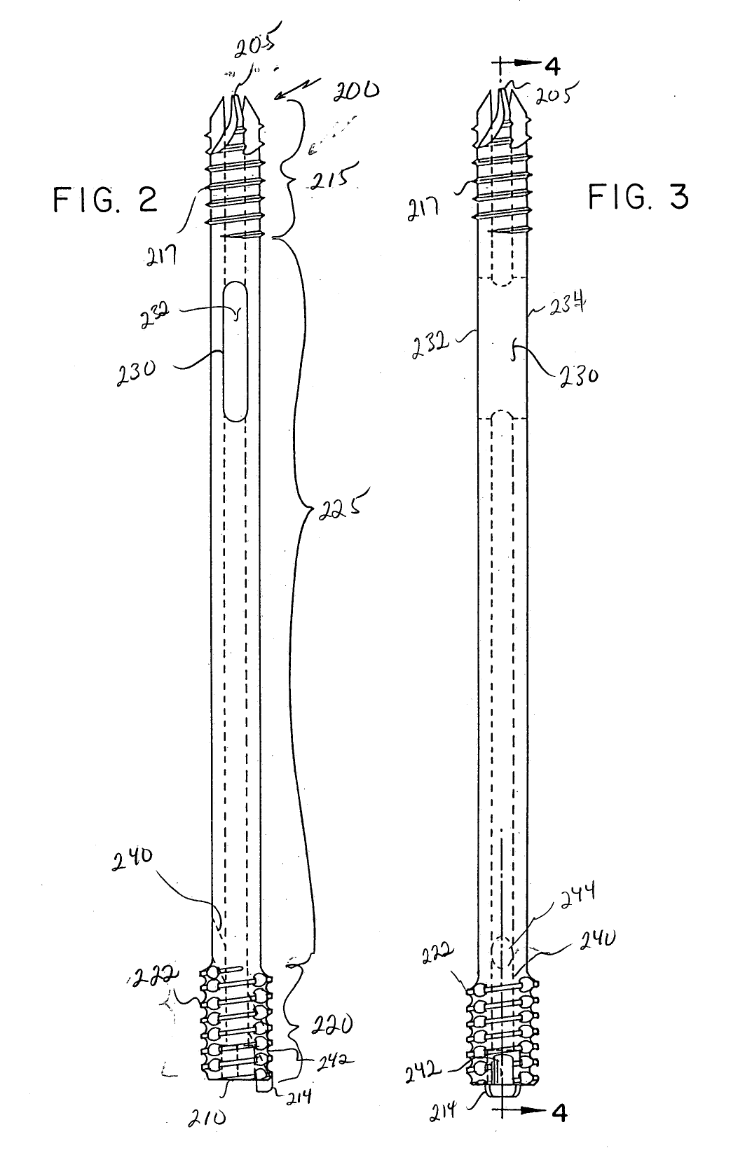

Referring now to FIGS. 2 and 3, a lockable double-threaded intramedullary compression screw 200, in accordance with the present invention, is depicted. As shown, the compression screw 200 has three distinct sections, which will also be sometimes referred to as portions. One is a narrower, e.g. smaller diameter, threaded section 215, which is located proximate to the leading end 205 of the compression screw 200 and has threads 217. The leading end 205 is the end that is first inserted into the cavity during surgery. Another is a wider, e.g. larger diameter, threaded section 220, which is located proximate to the trailing end 210 of the compression screw 200 and has threads 222. Still another is an unthreaded shaft section 225, which is located between threaded sections 215 and 220. The unthreaded shaft section 225 is preferably narrower, e.g. of smaller diameter, than both the threaded sections 215 and 220.

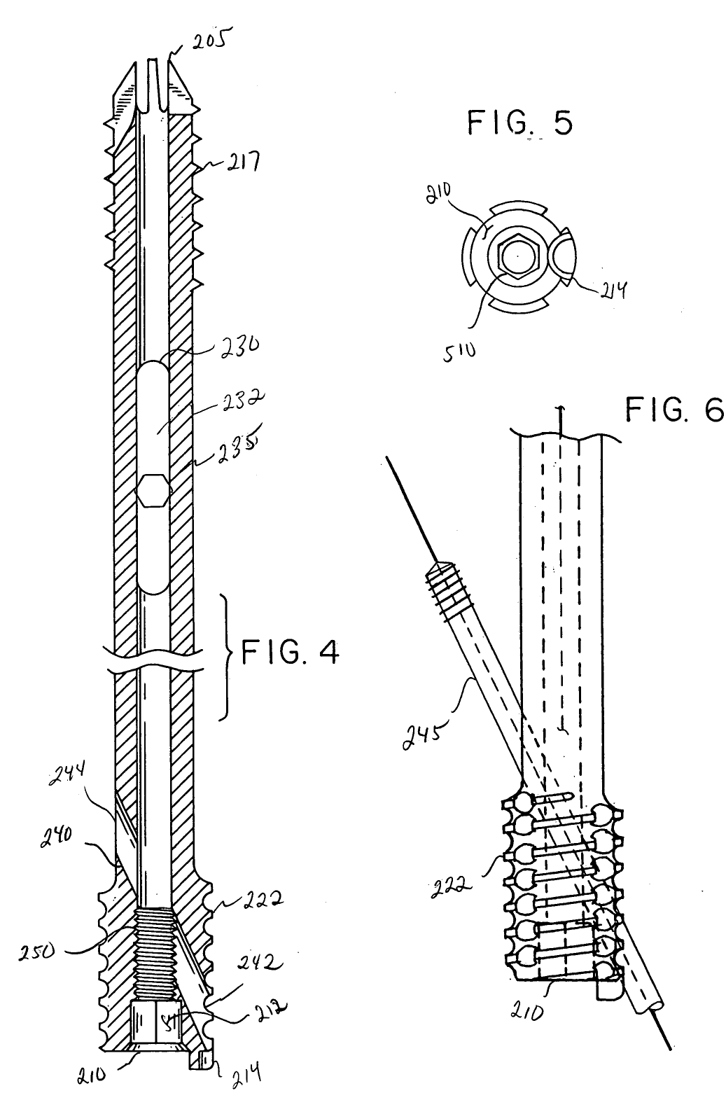

As shown in FIG. 5, the trailing end 210 of the compression screw 200 is provided with a socket aperture 510 to accommodate a tool (not shown), e.g. an Allren type wrench, for applying a clockwise torque to the compression screw 200. The application of the torque will initially cause the threads 217 to engage the bones in, and in the vicinity, of the joint. As the torque continues to be applied, the threads 222 will also engage the bones in the vicinity of the joint. This engagement of the threads and rotation of the compression screw 200 causes the compression screw to be drawn further into the cavity, passing through the bones in the joint to be fused.

Ultimately, the compression screw 200, will be drawn fully across the joint involved in the fusion, with the threads 217 engaged with the bone on the distal side of the joint and the threads 222 engaged with the bone on the proximal side of the joint, as shown in FIG. 1. The shape and size of the intramedullary canal, after appropriate reaming, ensures that the lockable double-threaded compression screw 200 fits firmly into the intramedullary canal when placed therein over a well-placed guide wire, as is well understood by those skilled in the art.

The placement of the double-threaded compression screw 200, after proper reaming, over a guide wire in the intramedullary area of the involved bones, results in the application of compressive forces across the involved joints, because of the double threading of the compression screw 200. As described above, the leading threads 217 fix themselves in the intramedullary area of the distal bone involved in the fusion, e.g. the tibia, fibula and/or talus. The trailing threads 222 pull, coapt and compress the proximal bone or bones, e.g. the calcaneus and talus, together to form a solid fusion mass of one bone.

The addition of locking screws 235 and 245, as shown in FIGS. 4 and 6, proximally via through-hole 240 and distally via slotted through-hole 230, prevents the loss of coaptation and compression. The locking screws 235 and 245 also remove the influence of any torsion forces. Preferably, the through-holes 230 and 240 are sized to accommodate a 4 mm or larger locking screw, and accordingly the locking screws 235 and 245 have a threaded diameter at least 4 mm.

The through-hole 240 extends along a straight longitudinal axis between a first opening 242, in a first area of an outer periphery of the compression screw 200 proximate to the trailing end portion 220, and a second opening 244, in a second area of the outer periphery of the compression screw 200 which is further from the trailing end 210 than the first opening 242. As shown, the second opening 244 is in the unthreaded shaft portion 225 of the compression screw 200. However, it will be recognized that in some implementations, the second opening 244 could be located in the trailing end portion 220 of the compression screw 200. The longitudinal axis of the through hole 240 intersects the substantially straight longitudinal axis of the compression screw 200 at an angle of other than 90 degrees. Most typically, this angle will be 45 degrees or less.

Extending from the trailing end 210, and away from the leading end of the compression screw 200, is a sleeve portion 214. The sleeve portion 214 is aligned with the opening 242 of the through-hole 240 and is designed to guide the locking screw 245 into the first opening 240.

The through-hole 230 extends along a straight longitudinal axis between a third opening 232, in a third area of the outer periphery of unthreaded shaft portion 225 of the compression screw 200, and a fourth opening 234i in a fourth area of the outer periphery of unthreaded shaft portion 225 of the compression screw 200. Both the third and the fourth areas are proximate to the leading end 205 of the compression screw 200. The longitudinal axis of the through-hole 230 intersects the longitudinal axis of the compression screw 200. Most typically, this angle will be 90 degrees.

As shown, a cross section of the through-hole 230, taken along the its longitudinal axis, has a dimension, preferably of approximately 1 inch, in a direction parallel to the longitudinal axis of the compression screw 200 which is larger than a dimension in direction perpendicular to the longitudinal axis of the compression screw.

Referring to FIGS. 2-4, leading end portion 215 and the trailing end portion 220 of the lockable double-threaded compression screw 200 are axially spaced apart by the substantially cylindrical shaft portion 225. As depicted, the leading end portion 215 and the trailing end portion 220 have screw threads 217 and 222 that are similar, but not necessarily of the same pitch. The leading end 205 of the compression screw 200 is lodged into the proximal bone involved in the fusion and the threads 222 of the trailing end portion 220 of the compression screw are, for example, lodged in the inferior area and cortex of the calcaneus.

Preferably, the pitch of the threads 217 of the leading end section 215 is slightly greater than the pitch of the threads 222 of the trailing end section 220 and, as noted above, the external diameter of the trailing end portion 220 of the compression screw 200 is greater than the diameter of the leading end portion 215. However, it should be understood that the relationships between pitch of the threads 217 and 222 and the diameter of the leading and trailing end section 215 and 220 can be varied as may be desired for the particular implementation.

As previously discussed, the central area of the trailing end 210 has an aperture 510, which may be in the form of a slot, disposed in the head 212 of the compression screw 200. As shown, the head 212 forms part of the trailing end portion 220 of the compression screw 200. The aperture 510 is configured to accommodate an insertion tool (not shown), such as a wrench or screwdriver, for applying a clockwise torque to the compression screw 200. Application of the clockwise torques drives the compression screw 200 into the bone or bones involved in the fusion.

The aperture 510 may also accommodate an extraction tool (not shown), such as a wrench or screwdriver, for applying a counter-clockwise torque to the compression screw 200. The extraction tool and insertion tool may be the same tool or different tools. The application of the counter-clockwise torque drives the compression screw 200 from its position in the bone or bones involved in the fusion.

The compression screw 200 illustrated in FIGS. 1-6 is principally to be lodged in spongy inner tissue of a bone. The helical threads 217 and 222 create compression between the bones involved in the fusion.

Optionally, internal threads 250 are provided beyond the aperture 510 in the head 212 of the compression screw 200. The internal threads 250 are formed within the trailing end portion 220 of the compression screw 200. These threads are adapted to accommodate a threaded extraction tool (not shown). The threads of the threaded extraction tool will engage the internal threads 250 as the threaded extraction tool is turned counter-clockwise, thereby locking the threaded extraction tool to the compression screw 200. After locking, the threaded extraction tool can be used to apply a further a counter-clockwise torque to the compression screw 200. The application of the counter-clockwise torque, after locking, drives the compression screw 200 from its position in the bone or bones involved in the fusion. Furthermore, because the threads of the threaded extraction tool are in locked engagement with the internal threads 250, the threaded extraction tool can also be used to apply a tension force to the compression screw 200. The application of the tension force, after locking, may be sufficient to pull the compression screw 200 from its position in the bone or bones involved in the fusion.

The use of the compression screw 200, and in particular the extraction of the compression screw 200 from the bones involved in the fusion, is greatly facilitated by the accommodation of an extraction tool that can be locked into the trailing end portion 220 of the compression screw 200 by counter-clockwise engagement with the internal threads 250, via the trailing end 210 of the compression screw.

In using the double-threaded compression screw 200, it is first necessary to select a compression screw suited to the task at hand, and to then follow the insertion procedure described above. The selection of the screw can be done by measuring preoperative x-ray images of the bones involved.

The foregoing description of the present invention is illustrative and explanatory. Various changes in size, shape and materials, as well as in the details of the preferred embodiment of the illustrated construction thereof may be made without departing from the spirit of the invention.

Referring again to FIG. 1, an anterior longitudinal midline approach to the ankle was used to approach the joint and to debride the cartilage from the distal tibia and the proximal talus. Next, a 1 inch transverse plantar incision was made at the intersection of a line drawn along the anterior border of the fibula proceeding along the plantar surface of the foot with a line drawn to the plantar center of the heel. Blunt dissection was made down to the inferior surface of the calcaneus. A periosteal elevator was used to gently push the soft tissue from the proposed entry site for an appropriate 3/32 guide wire to be centered through the intersection process of the heel and through the calcaneus, through the talus and into the intramedullary area of the distal tibia. While the guide wire was inserted, the foot was held in neutral position of flexion, extension, varus, valgus and rotation. The intramedullary position of the guide wire was verified by intraoperative xray images or fluoroscopy, and coaptation and alignment confirmed. A 7 mm reamer was used to prepare the path for the double-threaded compression screw being inserted over the guide wire. A 10 or 11 mm reamer was used to prepare the calcaneus. A compression screw of sufficient length, was selected, based on preoperative x-ray images of the involved bone. In this case the selected compression screw had a length of 6 inches. The double threaded screw must be long enough to engage the diaphysis of the tibia. The selected compression screw was driven over the guide wire through the calcaneus and into the intramedullary space of the distal tibia. Coaptation of the prepared surfaces of the distal tibia and proximal talus was rigid and immediate. Allograft was placed at the fusion site.

Using direct vision, a locking screw was passed over a guide wire into the proximal through hole, in the trailing end portion of the double-threaded compression screw. By palpation of the distal slot proximate to the leading end portion of the compression screw, a guide wire was inserted. The position of this guide wire passing through the distal slot in the leading end portion of the double-threaded screw was confirmed anteriorly, posteriorly and laterally by fluoroscopy. The length of the guide wire was confirmed by measurement of the exterior length of the guide wire using a similar length guide wire. A locking screw of appropriate length was then selected and inserted over the guide wire. A torque was applied to the locking screw to thereby securely distally lock the compression screw in position. The wounds were irrigated thoroughly, more allograft bone was then placed around the fusion site, and the wounds were closed in the usual fashion. A sterile bulky dressing was applied and a well-padded short leg cast was then applied. Partial weight bearing on the lower extremity, using a walking aid, was allowed immediately.

1. An intramedullary lockable compression screw for stabilizing a joint in a body, comprising:
an elongated tubular member extending along a substantially straight first longitudinal axis between a leading end and a trailing end, and including (i) a threaded leading end portion of a first diameter disposed proximate to the leading end, (ii) a threaded trailing end portion of a second diameter, larger than the first diameter, disposed proximate to the trailing end, and (iii) an unthreaded shaft portion interconnecting the threaded leading end and the threaded trailing end portions; and
a through-hole configured to accommodate a locking screw, extending along a straight second longitudinal axis between a first opening, in a first area of an outer periphery of the tubular member proximate to the trailing end, and a second opening, in a second area of the outer periphery of the tubular member distal to the trailing end, with the second longitudinal axis intersecting the first longitudinal axis at an angle of other than 90 degrees.

2. An intramedullary lockable compression screw according to claim 1, wherein the through-hole is adapted to accommodate a 4 mm locking screw.

3. An intramedullary lockable compression screw according to claim 1, wherein:
the elongated tubular member has a sleeve portion extending from the trailing end, away from the leading end; and
the sleeve portion is aligned with the through-hole and configured to guide the locking screw into the first opening.

4. An intramedullary lockable compression screw according to claim 1, further comprising:
another through-hole configured to accommodate another locking screw, extending along a straight third longitudinal axis between a third opening, in a third area of the outer periphery of the tubular member, and a fourth opening, in a fourth area of the outer periphery of the tubular member, the third and the fourth areas being proximate to the leading end of the tubular member, with the third longitudinal axis intersecting the first longitudinal axis.

5. An intramedullary lockable compression screw according to claim 4, wherein the third longitudinal axis intersects the first longitudinal axis at a 90 degree angle.

6. An intramedullary lockable compression screw according to claim 4, wherein the other through-hole is adapted to accommodate a 4 mm locking screw.

7. An intramedullary lockable compression screw according to claim 4, wherein a cross section of the other through-hole, taken along the third longitudinal axis, has a first dimension in a direction parallel to the first longitudinal axis and a second dimension, smaller than the first dimension, in direction perpendicular to the first longitudinal axis.

8. An intramedullary lockable compression screw according to claim 7, wherein the first dimension is approximately 1 inch.

9. An intramedullary lockable compression screw according to claim 1, further comprising:
an aperture in the trailing end of the tubular member, configured to accommodate a tool for applying a torque to the tubular member.

10. An intramedullary lockable compression screw according to claim 9, wherein the aperture in the trailing end of the tubular member, includes:
a non-threaded portion configured to accommodate a tool for applying a torque to the tubular member; and
a threaded portion configured to engage a tool for extracting the tubular member from a body joint.
